(19) United States
(12) Patent Application Publication
(10) Pub. No.: US 2009/0135992 A1
(43) Pub. Date: May 28, 2009

(54) METHOD FOR THE PROCESSING OF RADIOGRAPHY CARDIAC IMAGES WITH A VIEW TO OBTAINING A SUBTRACTED AND REGISTERED IMAGE

(76) Inventors: Regis VAILLANT, Sur Yvette (FR); Francois KOTIAN, Villepreux (FR); Elisabeth SOUBELET, Meudon (FR)

Correspondence Address: General Electric Company;GE Global Patent Operation, PO Box 861, 2 Corporate Drive, Suite 648, Shelton, CT, 06484, US

(21) Appl. No.: 12/269,952

(22) Filed: Nov. 13, 2008

(30) Foreign Application Priority Data
Nov. 27, 2007 (FR) 0759344

(51) Int. Cl. A61B 6/00; G06K 9/00

(52) U.S. Cl. 378/4; 382/130

(57) ABSTRACT

In a method for processing radiography cardiac images in order to obtain a subtracted and registered fusion image, at the start of a medical intervention, a radiology image of the heart is obtained by injection of a contrast product into one of the anatomical structures of the heart. Radioscopy images are acquired and are viewed in real time. A subtraction algorithm is implemented. This subtraction algorithm uses the radiology images to make the anatomical structures of the heart visible in the radioscopy images. A registration algorithm is implemented. This algorithm is capable of registering these two images by means of a catheter placed in the coronary sinus. This registration makes it possible to obtain perfect concordance between the two subtracted images.

### CROSS-REFERENCE TO RELATED APPLICATIONS

This application claims priority under 35 U.S.C. § 119(a)-(d) to prior-filed, co-pending French patent application serial number 0759344, filed on Nov. 27, 2007, which is hereby incorporated by reference in its entirety.

### BACKGROUND OF THE INVENTION

1. Field of the Invention

Embodiments of the invention find advantageous but not exclusive application field of medical imaging and more particularly in X-ray imaging in cardiology. The present Embodiments of the invention provide a method for the processing of radiography heart or cardiac images in order to obtain a subtracted and registered image. Embodiments of the invention further provide also relates to a station for the reviewing of medical images comprising a software program for the implementing of such an image-processing method.

2. Description of Related Art

X-ray imaging is presently very widely used for the diagnosis and treatment of heart pathologies. The treatment may be inter alia electrophysiological treatment.

In a certain number of interventional procedures or operations relating to the improvement of the electrical conduction of the heart, the practitioner needs to handle catheters and/or guides within the cavities of the heart. These interventions are used to perform different procedures such as, for example, ablation of auricular fibrillation or biventricular stimulation.

Ablation of auricular fibrillation is used to eliminate a problem of heart rate characterized by rapid, desynchronized and totally inefficient auricular contractions caused by disordered electrical activity of the muscular fibers of the auricles. Biventricular stimulation resynchronizes the heart during cardiac insufficiency.

These techniques are used to avert the need for heavy surgical interventions.

During the interventional procedure, the progress of the handling of the catheter is viewed and controlled by means of X-rays. However, anatomical structures of strategic importance such as the left auricle and the pulmonary veins in the case of a surgical procedure for ablation of auricular fibrillation, and the coronary sinus and its branches in the case of a procedure of biventricular stimulation, are not represented by X-ray apparatuses because they show no contrast relative to the surrounding anatomical structure.

For all these applications, knowledge of anatomical data would be very useful during the intervention in order to locate the tools or catheters relative to these structures.

A recent approach consists of the production, at the beginning or before the medical intervention, of a 3D image using a system of computer tomography or magnetic resonance or by rotation of the X-ray system. This approach comprises means capable of registering the pre-interventional 3D images with projection images of the radiography system so as to be subsequently fused with them. This fusion enables the practitioner to view at the same time the intervention tool and the anatomy. This type of approach is widely processed in the prior art.

The drawback of this approach is that it requires a pre-interventional 3D image and that concordance much be achieved between the acquisition geometries of the 3D image and the 2D image during the registration.

### BRIEF SUMMARY OF THE INVENTION

Embodiments of the invention address the drawbacks of the techniques explained above. To this end, an embodiment of the invention provides a method for the processing of radiography cardiac images. This method is used to make the anatomical structures of the heart visible on radioscopy images viewed in real time without using a pre-interventional 3D image.

Thus, one embodiment of the invention requires radiology images of the heart at the beginning of the medical intervention. To obtain these images, a contrast medium is injected into an anatomical structure of the heart that is to be made visible relative to the intervention to be performed. This contrast medium makes these anatomical structures opaque to X-rays. These images are obtained with a standard X-ray intensity.

An embodiment of the invention is configured to acquire radioscopy images that are viewed in real time. These images are obtained with X-ray intensity reduced by about ten times relative to the intensity of X-rays of radiology images.

An embodiment of the invention is configured to implement subtraction algorithm that uses the radiology images with contrast medium previously acquired during the medical intervention to make the anatomical structures of the heart visible on the radioscopy images. To this end, the algorithm is capable of subtracting the radioscopy images obtained without any injection of contrast medium from the radiography images obtained with injection of contrast medium. This subtraction gives the practitioner a final image that is more appropriate to the intervention.

However, the subtraction obtained does not enable the practitioner to perform the intervention with precision. Indeed, the main problem encountered in the specific part of the anatomy, namely the heart, is that it undergoes major movements due to the heart cycle and the patient's respiration.

An embodiment of the invention is configured to implement an algorithm of registration capable of eliminating these movements by registering all the images acquired by means of a catheter placed in the coronary sinus. This registration makes it possible to obtain a subtracted image showing the practitioner, at the same time, the intervention tool and the structure of the anatomy into which the contrast medium has been injected.

An embodiment of the invention is thus particularly well suited to a procedure of ablation in which the ablation catheter will be used to “burn” the tissue of the cardiac wall in order to modify the electrical conduction on the surface of the wall. Indeed, when a registration is done during the subtraction of the radiology images with contrast medium from the radioscopy images without contrast medium, these images are brought into concordance. Consequently, pulmonary veins and other zones implicated in the launching and tracking of an auricular fibrillation may be identified more precisely and more simply on the radioscopy images. This improves the rate of success of a procedure of ablation by catheter.

The embodiments of the invention also enable the practitioner to view the coronary sinus and the left ventricle on the radioscopy images, thus enabling the stimulation electrodes to be made to navigate and to be placed at the position that is most appropriate in the case of biventricular stimulation procedures.

The fact of having perfect concordance between the two subtracted images enables for example a cardiologist to achieve real-time tracking of the progress of the catheters during an intervention.

Embodiments of the invention make it possible to use the device placed at a fixed position, such as the catheter of the coronary sinus, in a moving organ, in this case the heart, to track the movement of this organ and then apply standard angiography approaches.

In an embodiment, a method for the processing of cardiac images in which:

at the start of a medical intervention, a contrast medium is injected into an anatomical structure of a patient's heart,
the patient's heart is exposed to radiation produced by a radiography imaging apparatus,
through a detector, a radiology image with contrast medium representing an internal structure of the heart is obtained,
during the medical intervention, the patient's heart is exposed to radiation produced by the radiography imaging apparatus,
through the detector, a succession of radioscopy images representing an internal structure of the heart is obtained,
the method comprising:
applying an algorithm for the subtraction of images between the radiology image with contrast medium and the radioscopy images,
performing a registration between the radiology image with contrast medium and the radioscopy images to obtain a fusion image;
synchronizing the fusion image with an electrocardiogram signal; and
displaying the synchronized fusion image on a display screen.

Advantageously, an embodiment of the invention is characterized in that synchronizing the fusion image comprises:

during the acquisition of the radiology image with the contrast medium, synchronizing said image with the electrocardiogram,
during the acquisition of the radioscopy images, synchronizing said images with the electrocardiogram.

Advantageously, an embodiment of the invention is characterized in that subtracting the images comprises:

subtracting each pixel gray level value of the radiology image with contrast medium from the corresponding pixel gray level value in the radioscopy images, the correspondence being determined by the position of the pixels in these images.

Advantageously, an embodiment of the invention further comprises recomputing the subtraction whenever the imaging apparatus or the patient is in motion.

Advantageously, according to an embodiment of the invention, the registration of the radioscopy images with the radiology image comprises:

applying an algorithm for detecting a catheter of the coronary sinus to the radioscopy images and to the radiology image with contrast medium,
wherein the registration is done by superimposing the catheter of the coronary sinus detected in the radioscopy images and the catheter of the coronary sinus detected in the radiology image with contrast medium.

Advantageously, according to an embodiment of the invention, the registration is done by putting the external contours of the anatomical structures in the radioscopy images and those of the radiology image with contrast medium into concordance with each other.

Advantageously, according to an embodiment of the invention, the registration is done by achieving the concordance, in the radioscopy images and in the radiology image with contrast medium, of an artificial object introduced into the heart with the aim of identifying the motion of the anatomical structure of said heart.

Advantageously, according to an embodiment of the invention, the anatomical structure into which a contrast medium is injected is determined as a function of the medical intervention to be performed.

Advantageously, according to an embodiment of the invention, the registration is recomputed or redone manually whenever the imaging system or the patient is in motion.

Advantageously, according to an embodiment of the invention, the radiology image with contrast medium and the radioscopy images are segmented, this segmentation being done manually or automatically.

Advantageously, according to an embodiment of the invention:

markers are created in the fusion image at predetermined positions representing an anatomical position or a position of a zone to be processed.

Embodiments of the invention also provide an X-ray apparatus for implementing said method for processing radiography cardiac images in order to obtain a subtracted and registered image.

### BRIEF DESCRIPTION OF THE DRAWINGS

Embodiments of the invention will be understood more clearly from the following description and the accompanying figures. These figures are given purely by way of an indication and in no way restrict the scope of the invention.

FIG. 1 shows an apparatus for the production of radioscopy and radiology images implementing an image-processing method according to an embodiment of the invention.

FIG. 2 illustrates means implementing the method according to an embodiment of the invention.

### DETAILED DESCRIPTION OF EMBODIMENTS OF THE INVENTION

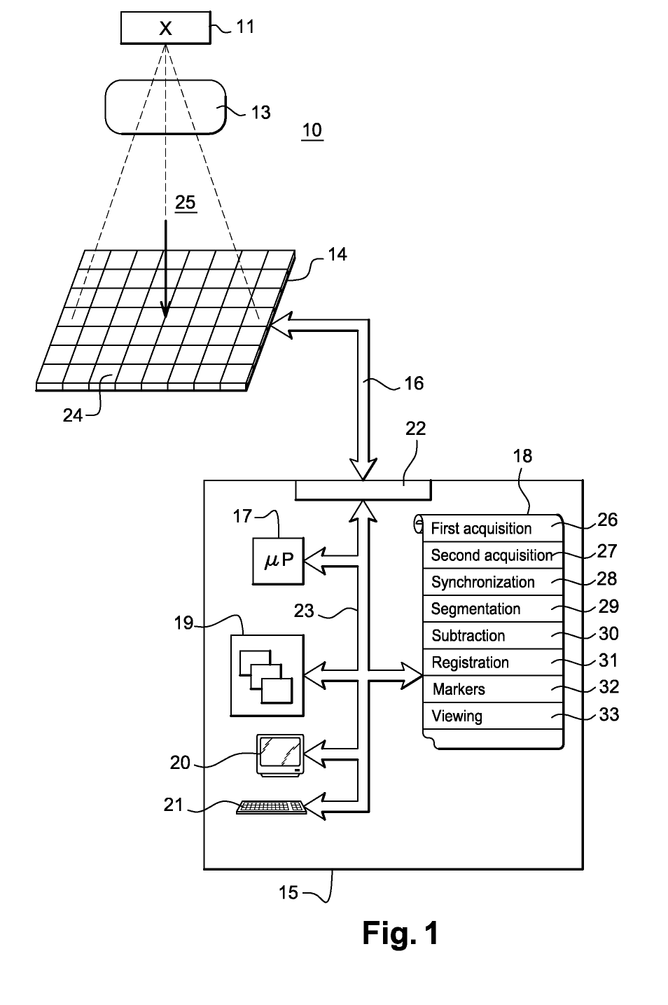

FIG. 1 illustrates an apparatus for the production of radioscopy images implementing a cardiac image processing method according to an embodiment of the invention. The radioscopy machine is a medical imaging apparatus used to obtain a dynamic radiological image, namely a sequence of images or a video sequence of the patient. This tool is used for diagnostics and for interventions i.e. during the treatment of the patient, in assisting with an intervention on the patient. The radioscopy machine is used here to help in an intervention performed by the practitioner in the patient's heart.

The images produced by the X-ray production apparatus 10 result from the detection of an incident irradiation coming from a radiation source 11 to which a patient 13 is exposed. The apparatus 10 also includes an image detector 14 and a control circuit 15.

The image detector 14 sends out electrical signals corresponding to the energy of the rays received. These electrical signals are transmitted to the control circuit 15 by means of an external bus 16. The image detector 14 is thus electrically coupled to the control circuit 15.

The electrical signals enable a circuit 15 to produce an image corresponding to the part of the body analyzed. These images may be viewed by means of a screen of this circuit 15, or printed, or stored.

In one example, the circuit 15 has a microprocessor 17, a program memory 18, a data memory 19, a display screen 20, a keyboard 21 and an input/output interface 22, these elements being interconnected by an internal bus 23.

The detector 14 has a multitude of detection zones or pixels 24 arranged in two dimensions. The first dimension is the x axis and the second dimension is the y axis. The image is distributed into rows and columns corresponding to a matrix sized (n×m). The control circuit 15 enables measurement of a load created in each pixel of the respective detector 14, in response to an incident irradiation. The dimensions of the image processed by the circuit 15 are preferably the same as those of the initial image. They are those of a matrix, in one example a matrix of 1024×1024 pixels, 512×512 pixels or 256×256 pixels or any other size, or even a non-square matrix. These dimensions are not restrictive and may be changed for the requirements of the invention.

When a radiology exposure is taken, a dose 25 of radiation is sent by the radiation source 11 to the patient's body 13. This dose 25 goes through the patient's body 13 and is received by the image detector 14.

In the present description, actions are attributed to apparatuses or programs. This means that the actions are executed by a microprocessor of this apparatus or an apparatus comprising the program, said microprocessor then being controlled by instruction codes recorded as a program in a memory of the apparatus. These instruction codes are used to implement the resources of the apparatus and therefore to carry out the action undertaken.

The program memory 18 is divided into several zones, each zone corresponding to a function or a mode of operation of the program of the control circuit 15.

Depending on the variants of the invention, the memory 18 comprises several zones. A zone 26 comprises instruction codes for the acquisition, in a first acquisition, of at least one radiology image with contrast injection.

A zone 27 comprises instruction codes for the acquisition, in a second acquisition and in real time, of a succession of radioscopy images. A zone 28 comprises instruction codes to synchronize the radiology images with the contrast medium and the radioscopy images with an electrocardiogram signal. A zone 29 comprises instruction codes to segment the images acquired at the zones 26 and 27.

A zone 30 comprises instruction codes to process these two acquisitions of images in applying an image subtraction algorithm. A zone 31 comprises instruction codes to perform a registration between these two image acquisitions.

A zone 32 comprises instruction codes to create markers at an anatomical position or at a position of the zone to be processed of the image obtained after subtraction and registration. These markers may be a graphic annotation, especially a triangle, a square, or even a letter or a figure. They may also be a binary number. These markers may be obtained by a coloring of said position. They are used in this case for a color imaging of their location. These markers enable the practitioner to know the zones to be processed and the zones already processed. This enables the practitioner to avoid processing a same zone several times. A zone 33 comprises instruction codes to view the final registered and subtracted image.

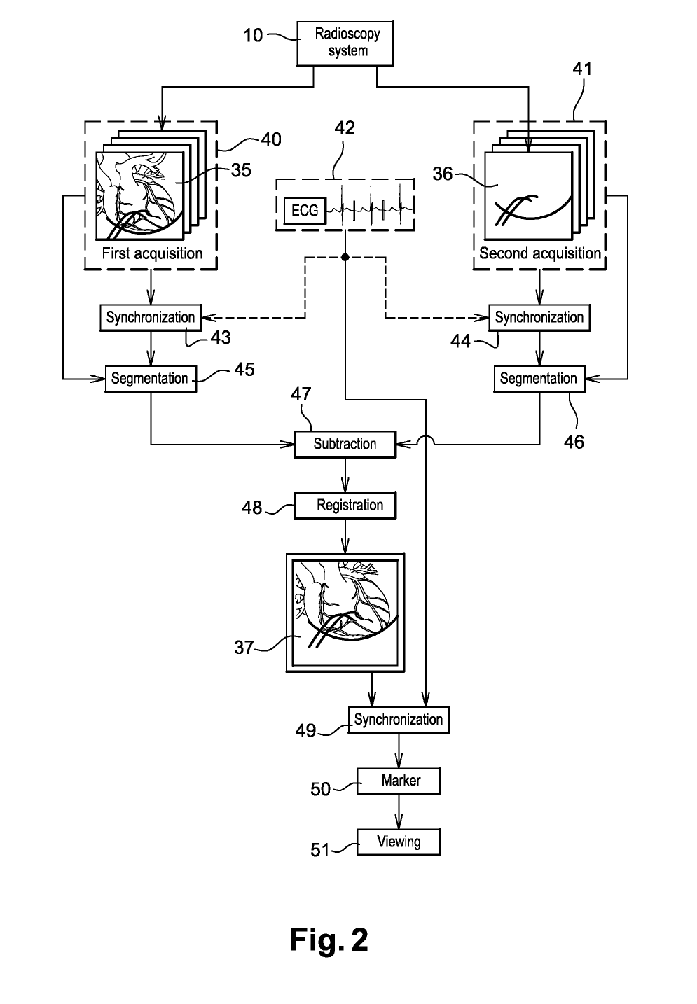

FIG. 2 illustrates means for implementing an embodiment of the method of the invention for processing heart or cardiac images. The example of FIG. 2 can be used in a medical intervention procedure such as for example a procedure for the ablation of auricular fibrillation or a biventricular procedure.

At the beginning of a medical intervention, a first acquisition of radiography images is done in a preliminary step 40. During the first acquisition, a contrast medium is injected into one of the anatomical structures of the heart. This anatomical structure is determined as a function of the medical intervention to be performed.

This contrast injection may have been done for example in one of the cavities of the heart. It can also be done especially in the right ventricle or right auricle. The contrast medium is generally an iodized compound. It makes the anatomical structures opaque to X-rays.

The control circuit 15 acquires at least one radiology image 35 of the heart with the injected contrast medium and with standard X-ray intensity.

During the medical intervention, a second acquisition of radiography images is performed at the following step 41. At the step 41, the circuit 15 acquires a succession of radioscopy images 36. These radioscopy images 36 are obtained with a smaller dose of X-rays than the dose of X-rays given to obtain the radiology image of the step 40. In these radioscopy images 36, the anatomical structures of the heart are not visible.

In the step 42, the circuit 15 acquires an electrocardiogram (ECG) signal.

According to one embodiment of the invention, synchronization steps 43 and 44 are executed respectively during the first and second operations of image acquisition. According to this embodiment, in the step 43, simultaneously with the first acquisition of the radiology image 35, the electrocardiogram (ECG) signal is acquired in order to obtain the radiology images 35 synchronized with the ECG signal. Similarly, at the step 44, simultaneously with the second acquisition of the radioscopy images 36, the electrocardiogram (ECG) signal is acquired in order to obtain radioscopy images 36 synchronized with the ECG.

The synchronization of these radiography images 35 and radioscopy images 36 with the electrocardiogram signal enables the motion due to the cardiac cycle to be separated from these images.

At a step 45 following the step 43, the radiology images 35 are segmented. This segmentation is aimed at separating the objects from one another and from the background in the images in extracting the contours or in carrying out the segmentation into homogenous regions. This segmentation enables the extraction of the cavities of the heart only from the radiology image 35. This segmentation can be done manually or automatically. At a step 46 following the step 44, the radioscopy images 36 are also segmented.

The steps 40, 43 and 45 are performed at the beginning of an intervention.

At a step 47, following the segmentation steps 45 and 46, a subtraction technique is applied to the radiology image 35 and to the radioscopy image 36 obtained at the instant t. This subtraction technique is a combination of a superimposition of two images to demonstrate the differences between them. This subtraction enables the performance of an image reconstruction in which, from each gray level value of a pixel of the radiology image 35, the corresponding pixel gray level value in the radioscopy image 36 is subtracted. This correspondence is determined by the position in the plane (x, y) of the pixels in these images.

This subtraction highlights the different elements of the image to be processed more efficiently. This subtraction is recomputed whenever the apparatus or the patient is in motion. The motions of the apparatus are either shifts of a table on which the patient is reclining or low-amplitude rotations of a C arm to which the detector is fixed.

At a following step 48, a preferably automatic registration of the radiology images 35 is done with the radioscopy image 36. The registration can be done or adjusted manually by a user. This registration is redone or recomputed whenever the apparatus or the patient is in motion.

To obtain a precise registration, it is not necessary to define a common reference such as the acquisition geometry. Indeed, the two image acquisitions are obtained with the same X-ray apparatus and with the same orientation.

The registration is preferably done by a superimposition of a coronary sinus catheter whose characteristic reversed j shape is quite visible in the radioscopy images 36 and radiology images 35.

It is practical to choose a catheter of this kind to perform the registration. Indeed, in almost all medical interventions aimed at improving electrical conduction in the heart, a catheter is placed inside the coronary sinus for a continuous recording of the electrical activity of the heart at this central position. Consequently, such a catheter is almost always available.

Furthermore, the catheter placed in the coronary sinus has very high contrast relative to the anatomical structure and in principle is clearly visible in the radioscopy images 36. Consequently, the invention implements a detection algorithm capable of automatically locating this catheter in the radioscopy images 36. This detection algorithm can be implemented by directional filters capable of detecting the pixels of the catheter in the radioscopy images 36. These directional filters are applied to the radioscopy images 36.

Thus, this visible catheter is preferably used to register the radiology images 35 with contrast injection coming from the first acquisition with the radioscopy images 36 coming from the second acquisition.

In one variant, the registration of the step 48 can be implemented by placing the external contours of the anatomical structures in concordance.

In another embodiment, the practitioner may screw an electrode into the zone of interest, for example the inter-auricular or inter-ventricular septum. This electrode will then be used as a reference system throughout the procedure in order to register the masks injected with the current image.

The reference system used to implement this registration can be obtained by any artificial object introduced by the practitioner into the heart in order to identify the motion of the anatomical structure of said heart.

The result of the registration is a fusion image 37 of the radiology image 35 with, for example, the radioscopy image 36 obtained at the instant t.

In a preferred embodiment, the synchronization step is performed only after the registration step and not during the acquisition of the images. At a step 49 following the step 48, the control circuit performs synchronizes the fusion image 37 with the electrocardiogram signal. This synchronization enables the separation of the motion due to the cardiac cycle from the fusion image 37.

At a following step 50, markers may be added to the fusion image 37 in order to inform the practitioner of the anatomical position or the position of the zone to be processed in the image.

At a step 51 following the step 50, the fusion image 37 is viewed on the display screen 20. The subtraction and registration steps of the invention enable the insertion of a piece of critical anatomical information into the radioscopy images, even in a moving environment such as the heart.

The fusion image 37 can be used to facilitate a registration of the 3D models of anatomical regions of the heart with radioscopy images of these anatomical regions obtained with an X-ray apparatus. These 3D images of anatomical regions of the heart are produced at the beginning or before the medical intervention. They are acquired by means of computer tomography systems or magnetic resonance systems or by rotation of the system by X-rays.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the scope of the following claim.

1.-12. (canceled)

13. A method for processing cardiac images in which
at the start of a medical intervention, a contrast medium is injected into an anatomical structure of a patient's heart,
the patient's heart is exposed to radiation produced by a radiography imaging apparatus,
through a detector, a radiology image with a contrast medium representing an internal structure of the heart is obtained,
during the medical intervention, the patient's heart is exposed to radiation produced by the radiography imaging apparatus,
through the detector, a succession of radioscopy images representing an internal structure of the heart is obtained,
the method comprising:
subtracting images between the radiology image with the contrast medium and the radioscopy images;
performing a registration between the radiology image with the contrast medium and the radioscopy images to obtain a fusion image;
synchronizing the fusion image with an electrocardiogram signal; and
displaying the synchronized fusion image on a display screen.

14. The method of claim 13, wherein synchronizing the fusion image further comprises:
during the acquisition of the radiology image with the contrast medium, synchronizing said radiology image with the electrocardiogram,
during the acquisition of the radioscopy images, synchronizing said radioscopy images with the electrocardiogram.

15. The method of claim 13, wherein subtracting the images further comprises:
subtracting each pixel gray level value of the radiology image with the contrast medium from a corresponding pixel gray level value in the radioscopy images, the correspondence being determined by a position in the plane (x, y) of the pixels in these images.

16. The method of claim 13, wherein the subtraction is recomputed whenever the imaging apparatus or the patient is in motion.

17. The method of claim 13, wherein registering the radioscopy images with the radiology image further comprises:
applying an algorithm for detecting a catheter of a coronary sinus to the radioscopy images and to the radiology image with the contrast medium; and
superimposing the catheter of the coronary sinus detected in the radioscopy images and the catheter of the coronary sinus detected in the radiology image with the contrast medium.

18. The method of claim 13, wherein the registration is done by putting into concordance external contours of the anatomical structures in the radioscopy images with external contours of the anatomical structures of the radiology image with the contrast medium.

19. The method of claim 13, wherein the registration is done by achieving the concordance, in the radioscopy images and in the radiology image with the contrast medium, of an artificial object introduced into the heart to identify a motion of the anatomical structure of the heart.

20. The method of claim 13, wherein the anatomical structure into which a contrast medium is injected is determined as a function of the medical intervention to be performed.

21. The method of claim 13, wherein the registration is recomputed or redone manually whenever the imaging system or the patient is in motion.

22. The method of claim 13, further comprising:
segmenting the radiology image with the contrast medium and the radioscopy images.

23. The method of claim 13, further comprising:
creating markers in the fusion image at predetermined positions representing an anatomical position or a position of a zone to be processed.
